(19) United States
(12) Patent Application Publication
(10) Pub. No.: US 2009/0298884 A1
(43) Pub. Date: Dec. 3, 2009

(54) Esomeprazole Strontium Salt, Process For Its Preparation and Pharmaceutical Compositions Containing Same

(75) Inventors: Shekhar Bhaskar Bhirud, Navi Mumbai (IN); Nitin Sharad Chandra Pradhan, Thane (IN); Bobba Venkata Siva Kumar, Navi Mumbai (IN); Pravin Bhalchandra Kulkarni, Kalyan (IN)

Correspondence Address: M. CARMEN & ASSOCIATES, PLLC, 170 OLD COUNTRY ROAD, SUITE 400, MINEOLA, NY, 11501, US

(73) Assignee: GLENMARK GENERICS LTD., Andheri (East) Mumba (IN)

(21) Appl. No.: 11/920,024

(22) PCT Filed: May 2, 2006
(86) PCT No.: PCT/IB2006/001109

§ 371 Date: Jan. 16, 2009

Related U.S. Application Data
(60) Provisional application No. 60/682,991, filed on May 20, 2005.

(30) Foreign Application Priority Data
May 6, 2005 (IN) 564/MUM/2005

(51) Int. Cl. A61K 31/4439; C07D 401/02; A61P 1/00

(52) U.S. Cl. 514/338; 546/273.7

(57) ABSTRACT

A strontium salt of esomeprazole is provided. A process for preparing a strontium salt of esomeprazole is also provided comprising reacting esomeprazole free base or a sodium, potassium or lithium salt of esomeprazole with a strontium source in one or more solvents.

### PRIORITY

This application claims the benefit under 35 U.S.C. §119 to U.S. Provisional Application No. 60/682,991, filed May 20, 2005, and entitled “ESOMEPRAZOLE STRONTIUM SALT”, and to Indian Provisional Application No. 564/MUM/2005, filed May 6, 2005, and entitled “ESOMEPRAZOLE STRONTIUM SALT”, the contents of each of which are incorporated by reference herein.

### BACKGROUND OF THE INVENTION

1. Technical Field

The present invention generally relates to a novel strontium salt of esomeprazole, a process for its preparation and pharmaceutical compositions containing same.

2. Description of the Related Art

Omeprazole (also known as 5-methoxy-2-[[(4-methoxy-3,5-dimethyl-2-pyridyl)methyl]-sulfinyl]-1H-benzimidazole) is a well known compound used for treating diseases related to increased secretion of gastric acid because of an H⁺/K⁺ ATPase inhibitory action. The compound, being a sulfoxide, has an asymmetric center in the sulfur atom and may exist as a racemic mixture (a mixture of (R)-omeprazole and (S)-omeprazole). The optical isomers of omeprazole, particularly the (S) isomer, are believed to possess certain advantages over the racemic form.

The enantiomer (S)-omeprazole is commonly referred to as esomeprazole (also known as (5-methoxy-2-[(S)-[(4-methoxy-3,5-dimethyl-2-pyridyl)methyl]-sulfinyl]-1H-benzimidazole-1-yl) and is represented by the structure of formula I.

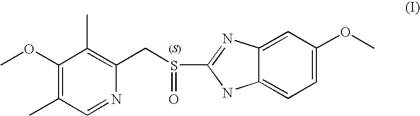

Esomeprazole is marketed in the United States as the magnesium trihydrate salt under the name Nexium® and is indicated for short-term treatment in the healing and symptomatic resolution of diagnostically confirmed erosive esophagitis. See, e.g., The Merck Index, Thirteenth Edition, 2001, pp. 1224-25, monograph 6913; and Physician's Desk Reference, “Nexium,” 60th Edition, pp. 645-649 (2005).

U.S. Pat. No. 4,738,974, herein incorporated by reference, discloses the lithium, sodium, potassium, magnesium and calcium salts of omeprazole.

U.S. Pat. No. 5,693,818, herein incorporated by reference, discloses optically pure salts of esomeprazole, such as sodium, potassium, lithium, magnesium, calcium and tetraalkylammonium salts.

Different forms of an active principle can have different bioavailability, solubility, stability, color, compressibility, flow ability and workability with consequent modification of the profiles of toxicological safety, clinical effectiveness and productive efficiency. Because improved drug formulations showing, for example, better bioavailability or better stability are consistently sought, there is an ongoing need for new or purer forms of existing drug molecules.

### SUMMARY OF THE INVENTION

In accordance with one embodiment of the present invention, a strontium salt of esomeprazole or a derivative thereof is provided.

In accordance with a second embodiment of the present invention, a process for preparing a strontium salt of esomeprazole is provided comprising reacting esomeprazole free base or a sodium, potassium or lithium salt of esomeprazole with a strontium source in one or more solvents.

In accordance with a third embodiment of the present invention, a pharmaceutical composition is provided comprising a therapeutically effective amount of a strontium salt of esomeprazole.

In accordance with a fourth embodiment of the present invention, a method for treating a gastric acid related condition is provided, the method comprising administering to a subject in need of treatment thereof a therapeutically effective amount of a strontium salt of esomeprazole.

### DEFINITIONS

The term “HPLC” as used herein means high performance liquid chromatograpy, and “e.e.” as used herein means enantiomeric excess.

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “buffering agent” as used herein means a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein means a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein means substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein means inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein means agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein means substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein means a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein means a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

The present invention is directed to a strontium salt of esomeprazole. The esomeprazole strontium salt can be of any form and can be obtained by at least reacting esomeprazole in its free base form or as a sodium, potassium or lithium salt of esomeprazole with a strontium source in a suitable solvent. Another aspect of the present invention provides a substantially pure strontium salt of esomeprazole. In one embodiment, a strontium salt of esomeprazole has a purity of greater than or equal to about 95%. In another embodiment, a strontium salt of esomeprazole has a purity of greater than or equal to about 98%. In yet another embodiment, a strontium salt of esomeprazole has a purity of greater than or equal to about 99%.

The esomeprazole free base or a sodium, potassium or lithium salt of esomeprazole to be used in the preparation of a strontium salt of esomeprazole of the present invention is well known and can be obtained by processes known in the art. See, e.g., U.S. Pat. Nos. 5,693,818; 5,948,789 and 6,162,816 and International Patent Application Nos. WO 92/08716, WO 98/54171 and WO 00/44744, the contents of each of which are incorporated by reference herein.

Suitable sources of strontium to react with the esomeprazole free base or a sodium, potassium or lithium salt of esomeprazole include, but are not limited to, inorganic acid salts of strontium, organic acid salts of strontium, and the like and combinations thereof. Suitable inorganic acid salts of strontium for use in the process of the present invention include, but are not limited to, strontium chloride, strontium chloride hexahydrate, strontium nitrate, strontium bromide, strontium sulfate, strontium carbonate and the like and mixtures thereof. Suitable organic acid salts of strontium for use in the process of the present invention include, but are not limited to, strontium acetate, strontium isopropoxide, strontium oxalate, strontium tartrate and strontium succinate and the like and mixtures thereof. The amount of the strontium source will ordinarily range from about 1 molar equivalent to about 5 molar equivalents per molar equivalent of esomeprazole starting material.

Soluble solvents for use herein include any solvent or solvent mixture in which the esomeprazole free base or salt is soluble. Representative examples of such solvents include, but are not limited to, water, alcohols, ketones, cyclic ethers, chlorinated hydrocarbons, nitrites, dipolar aprotic solvents and the like, and mixture thereof. Examples of alcohols include methanol, ethanol, isopropanol and the like. Examples of ketones include acetone, methyl isobutyl ketone and the like. Examples of cyclic ethers include tetrahydrofuran, dioxane and the like. Examples of chlorinated hydrocarbons include methylene dichloride, ethylene dichloride and the like. Examples of nitrites include acetonitrile and the like. Examples of dipolar aprotic solvents include dimethylsulfoxide, dimethylformamide and the like. Mixtures of all of these solvents are also contemplated.

In general, the reaction can be carried out at a suitable temperature and for a sufficient period of time to form a strontium salt of esomeprazole. A suitable temperature will ordinarily range from about 0° C. to about 50° C. In one embodiment, the temperature is an elevated temperature. In another embodiment, the temperature is room temperature. The time period can range from about 30 minutes to about 15 hours.

In one embodiment, the reaction involves reacting a first solvent solution containing at least esomeprazole free base or a sodium, potassium or lithium salt of esomeprazole and a first solvent with a second solvent solution containing at least a strontium source and a second solvent. The first and second solvents can be any of the aforementioned solvents. In one embodiment, the first and second solvents are the same. In another embodiment, the first and second solvents are different.

Following the reaction, the strontium salt of esomeprazole thus obtained is isolated and recovered. For example, when carrying out the reaction in a solvent which the reactants are relatively more soluble than the strontium salt of esomeprazole, e.g., water, the strontium salt forming reaction will be accompanied by a spontaneous precipitation out of solution of the esomeprazole strontium salt. The precipitated esomeprazole strontium salt can then be recovered by conventional techniques, e.g., filtration or centrifugation, optionally followed by washing and/or drying.

In another way of isolating strontium salt of esomeprazole, precipitation of the esomeprazole strontium salt may be facilitated by evaporating the solvent and/or by adding an anti solvent to the solution in the case where the solvent employed is a solvent in which the esomeprazole strontium salt is insoluble or sparingly soluble. In another embodiment, precipitation can also be induced by cooling the solution to a temperature sufficient to precipitate a solid of esomeprazole strontium salt, especially if the initial temperature during addition of the reactants is elevated. The precipitated esomeprazole strontium may then be recovered in a solid state by conventional techniques, e.g., filtration or centrifugation, optionally followed by washing and/or drying.

Suitable anti solvents that may be added to precipitate out a strontium salt of esomeprazole include, but are not limited to, lower alkyl ethers such as diethyl ether, diisopropyl ether and the like and mixtures thereof.

Yet another aspect of the present invention is directed to pharmaceutical compositions containing at least the novel strontium salt of esomeprazole of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The novel strontium salt of esomeprazole of the present invention also may be administered as suppositories, ophthalmic ointments and suspensions, and parenteral suspensions, which are administered by other routes. The dosage forms may contain the novel strontium salt of esomeprazole of the present invention as is or, alternatively, may contain the novel strontium salt of esomeprazole as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the novel strontium salt of esomeprazole of the present invention within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

In one embodiment, the novel strontium salt of esomeprazole for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. The term “micronization” used herein means any process or methods by which the size of the particles is reduced. For example, the particle sizes of the novel strontium salt of esomeprazole can be obtained by any milling, grinding, micronizing or other particle size reduction method known in the art to bring the solid state form of the novel strontium salt of esomeprazole of the present invention into any of the foregoing desired particle size range. As also used herein, esomeprazole strontium salt particles with reduced size are referred to as “micronized particles of esomeprazole strontium salt” or “micronized esomeprazole strontium salt”.

The esomeprazole strontium salt of the present invention may be used for inhibiting gastric acid secretion in mammals. In a more general sense, the esomeprazole strontium salt of the present invention may be used for the treatment of gastric acid-related diseases and gastrointestinal inflammatory diseases in mammals such as, for example, gastric ulcer, duodenal ulcer, reflux esophagitis, and gastritis. Furthermore, the esomeprazole strontium salt may be used for treatment of other gastrointestinal disorders where gastric antisecretory effect is desirable, e.g., in patients on NSAID therapy, in patients with gastrinomas, and in patients with accute upper gastrointestinal bleeding. The esomeprazole strontium salt may also be used in patients in intensive care situations, and pre- and postoperatively to prevent acid aspiration and stress ulceration. The esomeprazole strontium salt may also be used for treatment or prophylaxis of inflammatory conditions in mammals, including man, especially those involving lysozymal enzymes. Conditions that may be specifically mentioned are rheumatoid arthritis and gout. The esomeprazole strontium salt of the present invention may also be useful in the treatment of psoriasis as well as in the treatment of Helicobacter infections.

Actual dosage levels of the novel strontium salt of esomeprazole of the present invention may be varied to obtain an amount that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the novel strontium salt of esomeprazole of the present invention can be administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

The following examples are provided to enable one skilled in the art to practice the invention and are merely illustrative of the invention. The examples should not be read as limiting the scope of the invention as defined in the features and advantages.

### EXAMPLE 1

Preparation of Esomeprazole Strontium Salt

The potassium salt of esomeprazole (5 g) was dissolved in water (100 ml) at room temperature and a solution of strontium chloride hexahydrate (5 g) dissolved in water (100 ml) was slowly added. The reaction mixture was stirred at room temperature for about 1 to about 2 hours. The solids that precipitated were filtered and the wet cake was washed with water (25 ml). The wet cake was air dried at a temperature ranging from about 40° C. to about 45° C. to provide esomeprazole strontium salt (4.2 g). (HPLC Purity>99.6%,>99.5% enantiomeric excess (ee)).

### EXAMPLE 2

Preparation of Esomeprazole Strontium Salt

The sodium salt of esomeprazole (5 g) was dissolved in water (100 ml) at room temperature and a solution of strontium chloride hexahydrate (5 g) dissolved in water (100 ml) was slowly added. The reaction mixture was stirred at room temperature for about 1 to about 2 hours. The solids that precipitated were filtered and the wet cake was washed with water (25 ml). The wet cake was air dried at a temperature ranging from about 40° C. to about 45° C. to provide esomeprazole strontium salt (4.8 g). (HPLC Purity>99.3%,>99.8% ee).

### EXAMPLE 3

Preparation of Esomeprazole Strontium Salt

Esomeprazole free base (5 g) was dissolved in isopropanol (50 ml) at room temperature and a solution of strontium isopropoxide (5 g) dissolved in isopropanol (50 ml) was slowly added. The reaction mixture was stirred at room temperature for about 1 to about 2 hours and the reaction mass was quenched in water. The precipitated solids were filtered and the wet cake was washed with water (25 ml). The wet cake was air dried at a temperature ranging from about 40° C. to about 45° C. to provide esomeprazole strontium salt (4.0 g). (HPLC Purity>98.9%,>99.7% ee).

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

1. A process for preparing a strontium salt of esomeprazole, the process comprising reacting esomeprazole free base or a sodium, potassium or lithium salt of esomeprazole with a strontium source in one or more solvents.

2-5. (canceled)

6. The process of claim 1, wherein the strontium source is selected from the group consisting of an inorganic acid salt of strontium, organic acid salt of strontium and mixtures thereof.

7. The process of claim 1, wherein the strontium source is selected from the group consisting of strontium chloride, strontium chloride hexahydrate, strontium isopropoxide, strontium nitrate, strontium bromide, strontium sulfate, strontium carbonate, strontium acetate, strontium oxalate, strontium tartrate, strontium succinate and mixtures thereof.

8. The process of claim 1, wherein the amount of the strontium source is from about 1 to about 5 molar equivalents per molar equivalent of the esomeprazole free base or the sodium, potassium or lithium salt of esomeprazole.

9. The process of claim 1, wherein the solvent is selected from the group consisting of water, C₁-C₁₀ alcohol, C₁-C₁₀ ketone, C₁-C₁₀ cyclic ether, chlorinated hydrocarbon, nitrile, dipolar aprotic solvent and mixtures thereof.

10-13. (canceled)

14. The process of claim 1, further comprising precipitating the strontium salt of esomeprazole and recovering the strontium salt of esomeprazole.

15. The process of claim 1, comprising reacting a first solvent solution comprising esomeprazole free base or a sodium, potassium or lithium salt of esomeprazole and a first solvent with a second solvent solution comprising a strontium source and a second solvent.

16. (canceled)

17. The process of claim 15, wherein the first and second solvent are water or an alcohol.

18. The process of claim 15, wherein the first and second solvent are water or isopropanol.

19. (canceled)

20. The process of claim 15, further comprising precipitating the strontium salt of esomeprazole and recovering the strontium salt of esomeprazole.

21. (canceled)

22. The process of claim 20, wherein the step of precipitating the strontium salt of esomeprazole comprises adding an anti solvent to the reaction solution.

23-26. (canceled)

27. The process of claim 1, wherein the strontium salt of esomeprazole has a purity of greater than or equal to about 95%.

28-29. (canceled)

30. The process of claim 1, wherein the strontium salt of esomeprazole has an enantiomeric excess of greater than or equal to about 95%.

31-33. (canceled)

34. A strontium salt of esomeprazole or a derivative thereof.

35. The strontium salt of esomeprazole of claim 34, which is substantially.

36. (canceled)

37. The strontium salt of esomeprazole of claim 35, having a purity of greater than or equal to about 98%.

38. (canceled)

39. The strontium salt of esomeprazole of claim 34, having an enantiomeric excess of greater than or equal to about 95%.

40-41. (canceled)

42. A pharmaceutical composition comprising a therapeutically effective amount of the strontium salt of esomeprazole of claim 34.

43-46. (canceled)

47. The pharmaceutical composition of claim 42, wherein the strontium salt of esomeprazole is a micronized strontium salt of esomeprazole having a particle size of less than about 15 microns.

48-50. (canceled)

51. A method for treating a gastric acid related condition, the method comprising administering to a subject in need of treatment thereof a therapeutically effective amount of a strontium salt of esomeprazole of claim 34.

52. (canceled)
